(19) United States
(12) Patent Application Publication
(10) Pub. No.: US 2025/0156669 A1
(43) Pub. Date: May 15, 2025

(54) RFID SYSTEM AND METHOD FOR MONITORING THE DEGREE OF RIPENESS OF A PLURALITY OF FRUITS

(71) Applicant: ILIP S.R.L., Valsamoggia (BO) (IT)

(72) Inventors: Luigi GARAVAGLIA, Valsamoggia (BO) (IT); Filippo BIGNAMI, Valsamoggia (BO) (IT); Gaetano MARROCCO, Roma (IT); Cecilia OCCHIUZZI, Roma (IT); Nicola D'UVA, Roma (IT); Sara AMENDOLA, Roma (IT); Francesca CAMERA, Roma (IT)

(73) Assignee: ILIP S.R.L., Valsamoggia (BO) (IT)

(21) Appl. No.: 18/834,700

(22) PCT Filed: Feb. 25, 2022
(86) PCT No.: PCT/IT2022/000013

§ 371 Date: Jul. 31, 2024

(51) Int. Cl. G06K 19/07; G01N 33/02; G06K 19/077

(52) U.S. Cl. CPC G06K 19/0717; G01N 33/025; G06K 19/0723; G06K 19/07786

(57) ABSTRACT

A system and a method for monitoring the degree of ripeness of a plurality of fruits, in particular tropical fruits, are described. The system includes a support having a plurality of cavities for containing the fruits, an RFID tag operating in the UHF band, associated to each cavity for containing a fruit and arranged, in use, in contact with or close to the fruit, an interrogation device of the RFID tag having a reader and an interrogation antenna. The interrogation antenna is arranged in a seat of the support and is operatively associated to an RFID tag associated to a containing cavity, and means for processing electromagnetic signals (data) related to the degree of ripeness of the fruits coming from the reading of the RFID tags by the.

### FIELD OF APPLICATION

In its more general aspect, the present invention relates to the sector of fruit and vegetables.

In particular, the present invention relates to a system for monitoring the state or the degree of ripeness of a plurality of fruits, in particular but not exclusively tropical fruits, based on RFID technology. Said system comprises a support having a plurality of cavities for containing said fruits, at least one RFID tag operating in the UHF band, associated to each fruit and arranged in contact with or close to said fruit and at least one interrogation antenna operatively associated to at least one RFID tag, and means for processing electromagnetic signals (data) relative to or related to the degree of ripeness of said fruits coming from the interrogation of said RFID tags by means of said at least one interrogation antenna.

The present invention also relates to a method for monitoring a plurality of fruits that uses a system based on RFID technology as mentioned above.

The present invention further relates to a support suitable for the use in a system for monitoring the ripeness of a plurality of fruits as above, the support comprising a plurality of cavities for containing said fruits, one for each fruit, and at least one housing seat, preferably a plurality of housing seats, containing a respective interrogation antenna, each interrogation antenna being operatively associated to at least one RFID tag, preferably being associated to a group of RFID tags.

### Prior Art

In the sector of fruit and vegetables, the increasing demand for fruits, such as tropical fruits, meeting higher standards of quality and freshness, results in the need of monitoring the state or the degree of ripeness of the fruits from harvest and possible artificial ripening in designated ripening cells to the full distribution chain.

Traditionally, the state of ripeness of fruits is established on the basis of some characteristics of the fruit, such as for example soluble solids, pulp hardness, etc., which can be determined with simple instrumentation such as a refractometer, a penetrometer, etc. Said techniques, however, have the drawback of a poor representativeness and usually entail the destruction of the analyzed fruits.

More recently, non-destructive systems for monitoring the state of ripeness of fruits, such as tropical fruits, have been introduced, including in particular systems based on RFID technology.

As it is known, a RFID device comprises one or more RFID tags, commonly called RFID tags or transponders, which are intended to be attached to the object to identify or detect, and interrogation means such as a reader having an antenna capable of reading radio-frequency (RF) electromagnetic data (signals) transmitted by the RFID tags.

In particular, in the field of fruit monitoring, the RFID tag may be arranged in contact with or close to a respective fruit (for example in a package containing them as in the case of the so-called “smart” packaging), receives electromagnetic signals via the antenna of the reader and returns a backscattered signal affected by variations related to the state of ripeness of the fruit associated thereto.

It is in fact known that fruits have peculiar dielectric characteristics that change over time in connection to changes the fruit undergoes due to ripening (moisture, acidity, sugars) or to physiological alterations (texture) that occur during its storage.

Therefore, the passive antenna which is part of the RFID tags in contact with or close to the respective fruits accordingly modifies its matching impedance, acting as a transducer which sends a signal reflecting said modification.

The reader detects the electromagnetic signals reflected by the RFID tags in a passive way, such as the echoes of a conventional radar, and it can process said signals through suitable classification algorithms so as to decode the state of the fruits and classify them.

A system for monitoring the degree of ripeness of fruits based on RFID technology as mentioned above is described for example in patent EP 3648009 in the name of the Applicant.

Although the systems for monitoring based on RFID technology as illustrated above comply with the purpose they are conceived for, they have some limitations, including the fact that the accuracy of the measurements related to the degree of ripeness of fruits is limited and, moreover, said systems are not suitable for continuous measurements on a plurality of fruits.

The main purpose of the present invention is thus to provide a system and a method based on RFID technology for monitoring the state or degree of ripeness of a plurality of fruits which allow for a continuous analysis of the fruits to be monitored over a preset time period and, at the same time, a higher accuracy and/or precision of the electromagnetic measurements related to the state or degree of ripeness of said fruits, so as to overcome the limitations of the prior art described above.

Another purpose of the present invention is to provide a system and a method for monitoring the state or degree of ripeness of a plurality of fruits as above which are effective and reliable in the detection of the actual state of the fruit and which can be made and used with low costs.

### SUMMARY OF THE INVENTION

Said purposes are primarily achieved by a system for monitoring the degree of ripeness of a plurality of fruits, in particular tropical fruits, comprising a support having a plurality of cavities for containing said fruits, at least one RFID tag operating in the UHF band, associated to each cavity for containing a fruit and arranged, in use, in contact with or close to said fruit, an interrogation device of said at least one RFID tag comprising a reader and at least one interrogation antenna, said at least one interrogation antenna being arranged in a seat of said support and being operatively associated to at least one RFID tag associated to at least one containing cavity, and means for processing electromagnetic signals (data) relative to or related to the degree of ripeness of said fruits coming from the reading of said RFID tags by said interrogation device.

In an embodiment of the invention, the system comprises a plurality of RFID tags associated to each cavity for containing a fruit and arranged, in use, in contact with or close to said fruit.

In an embodiment of the invention, the plurality of RFID tags associated to each cavity for containing a fruit comprise a first RFID tag arranged, in use, in contact with or close to a basal area (or of the base) of said fruit, and at least one second RFID tag arranged, in use, in contact with or close to a side area, preferably an equatorial area, of said fruit.

In an embodiment of the invention, each containing cavity is delimited by a bottom and at least one side wall ending in an upper edge or opening with perimeter extension and, optionally, it has at least one tongue, preferably a plurality of tongues, extending from said upper edge or opening toward the inside of said containing cavity. In such embodiment, the plurality of RFID tags associated to each cavity for containing a fruit comprise a first RFID tag applied on the bottom of said containing cavity, and at least one second RFID tag applied on said at least one side wall and/or on said at least one tongue of said containing cavity.

In an embodiment of the invention, each cavity for containing a fruit comprises a plurality of said second RFID tags applied one after the other on at least one side wall and/or at least one tongue, preferably to one or more pairs opposite one another.

In an embodiment, said support comprises a base and a tray which are removably associated or coupled with each other, the tray comprising said cavities for containing respective fruits, and the base comprising at least one housing seat and a respective interrogation antenna housed in said seat, each housing seat with the respective interrogation antenna being arranged close to, preferably under, a respective group of cavities for containing fruits and each interrogation antenna being operatively associated to the RFID tags associated to the containing cavities of the respective group.

In an embodiment, the above-mentioned processing means are integrated in the reader, thereby constituting integral part thereof.

In an embodiment, the system according to the invention further comprises display means in communication with the processing means for displaying information about the degree or state of ripeness of fruits related to the data processed by the processing means.

The above-mentioned purposes are also accomplished by a method for monitoring the degree of ripeness of a plurality of fruits which uses a system as described above. Said method comprises the steps of:
providing at least one support as described above,
arranging a plurality of fruits in respective containing cavities of said at least one support with at least one RFID tag operating in the UHF band and associated to each containing pit, wherein said RFID tag is arranged, in use, in contact with or close to the respective fruit,
generating at least one electromagnetic signal in the UHF band by means of the reader of said interrogation device, each electromagnetic signal generated by said reader being irradiated/directed by an interrogation antenna so that it involves the RFID tag or the RFID tags of at least one cavity for containing a fruit which is/are operatively associated to said interrogation antenna,
recording, by means of said interrogation device, a plurality of signals transmitted by the RFID tags associated to the respective cavities for containing the fruits, each signal being related to the frequency and/or phase and/or power reflected (backscattered) by a RFID tag associated to a respective cavity for containing a fruit,
processing data relative to or related to the frequency and/or phase and/or power of the transmitted signals and/or processing data relative to or related to the variation of frequency and/or phase and/or power between the signals transmitted by the RFID tags and a preset value of frequency and/or phase and/or power,
displaying a piece of information about the degree or state of ripeness of said fruits related to the processed data.

In an embodiment, the method according to the invention further comprises:
comparing the processed data relative to or related to the frequency and/or phase and/or power of the signals transmitted by said RFID tags with reference signal data relative to or related to an initial state before the ripening of said fruits, and
classifying said fruits according to their state or degree of ripeness on the basis of the variation between said processed data and said reference data,
displaying a piece of information about the classification of said fruits.

The above-mentioned purposes are also achieved by a support suitable for monitoring the state or degree of ripeness of a plurality of fruits which may be used in a monitoring system as described above.

Said support comprises a plurality of cavities for containing fruits, one for each fruit, and at least one housing seat, preferably a plurality of housing seats, containing a respective interrogation antenna, each interrogation antenna being, in use, operatively associated to at least one RFID tag associated to a containing cavity or to a group of containing cavities.

In an embodiment of the invention, the support comprises at least one RFID tag associated to each cavity for containing a fruit and arranged, in use, in contact with or close to said fruit.

In an embodiment of the invention, the support comprises a plurality of RFID tags associated to each cavity for containing a fruit, said plurality of tags comprising a first RFID tag arranged, in use, in contact with or close to a basal area (or of the base) of said fruit, and at least one second RFID tag arranged, in use, in contact with or close to a side area, preferably an equatorial area, of said fruit.

In an embodiment of the invention, each containing cavity of the support has a bottom and at least one side wall ending in an upper edge or opening with perimeter extension and, optionally, at least one tongue, preferably a plurality of tongues, extending from said upper edge or opening toward the inside of said containing cavity, and a plurality of RFID tags associated to each cavity for containing a fruit, said plurality of tags comprising a first RFID tag applied on the bottom of said containing cavity, and at least one second RFID tag applied on said at least one side wall and/or on said at least one tongue of said containing cavity.

In an embodiment, the support comprises a base and a tray which are removably associated or coupled with each other, the tray comprising said cavities for containing respective fruits, and the base comprising at least one housing seat and a respective interrogation antenna housed in said seat, each housing seat with the respective interrogation antenna being arranged close to, preferably under, a respective group of cavities for containing fruits and each interrogation antenna being operatively associated to the RFID tags associated to the containing cavities of the respective group.

The above-mentioned purposes are also achieved by a stillage comprising a frame with vertical supporting elements, a plurality of shelves arranged between said supporting elements at predetermined heights between each other and connected to said supporting elements, and a plurality of supports as defined above arranged each on a respective shelf.

In an embodiment, said stillage is movable and comprises a plurality of small wheels fixed at the lower end of said vertical supporting elements and, optionally, at least one handle element, fixed at the upper ends of opposite supporting elements.

In an embodiment, the stillage comprises, integrated, a reader of the RFID tags associated to the containing cavities of the supports, said processing means (for example a computer) and said display means.

In another embodiment, the reader of the RFID tags associated to the containing cavities, the above-mentioned processing means and the above-mentioned display means are instead delocalized with respect to the stillage and are operatively connected to each other and to the supports containing the cavities for containing fruits during the use of the system according to the invention.

Further features and advantages of the present invention will result from the following description of some preferred embodiments, said description being given by way of a non-limiting example with reference to the accompanying figures.

### BRIEF DESCRIPTION OF THE FIGURES

In the figures:

FIG. 1 shows a perspective view of a tray of a support according to an embodiment of the present invention;

FIGS. 2-4 show respectively a top view and side views of the tray of the support of FIG. 1;

FIG. 5 shows a perspective view of a base of a support according to an embodiment of the present invention, said base being suitable to be coupled to the tray of FIG. 1;

FIGS. 6-8 show respectively a top view and side cross-section views of the base of the support of FIG. 1;

FIGS. 9-10 show each a view of a detail of a support according to another embodiment of the present invention;

FIG. 11 shows a side view of a stillage according to an embodiment of the invention;

FIG. 12 shows a top view of the stillage of FIG. 11;

FIG. 13 shows a cross-section view of the stillage of FIG. 11 according to the line A-A of FIG. 12;

FIG. 14 shows a cross-section view of the stillage of FIG. 11 according to the line B-B of FIG. 12;

FIG. 15 shows a schematic view of a detail of the system for monitoring the state or degree of ripeness of a plurality of fruits according to the invention;

FIG. 16 shows a schematic view of a detail of the monitoring system according to another embodiment of the invention.

### DETAILED DESCRIPTION

As mentioned previously, the monitoring system according to the invention comprises at least one RFID tag operating in the UHF band associated to each cavity for containing a fruit.

The RFID tag is by itself conventional and comprises a microchip and a RF antenna mounted on a substrate. In use, the substrate is used to attach the RFID tag directly on the fruit, or close to or in contact with it, in particular by fixing, for example with adhesives, the RFID tag in the cavity for containing said fruit.

The microchip has a printed circuit comprising an analog transponder for detecting and decoding a RF signal of interrogation and a decoder-processor for executing the RFID function and providing an analog-digital conversion for the communication with the transponder. The printed circuit further comprises a nonvolatile memory containing predetermined information which can be accessed using the decoder-processor.

Said predetermined information may be, for example, information related to the food (fruit) of application to be monitored, such as fruit type, origin, price, date of import and packaging.

The antenna of each RFID tag may be made of a flexible material, in particular of a metal material such as copper, aluminum or silver, and is operatively connected to the microchip. Advantageously, the RFID antenna may be made of a material suitable for the contact with food or it may be made suitable for the contact with food, for example by coating the material constituting the antenna with a film of a material suitable for food use, for example a film of a polymer material.

As known in the art, the printed circuit can recover the data stored by the memory and modulate an output RF signal by means of the transponder to transmit the data recovered to an interrogator, for example a reader. In addition to the recovery and the transmission of the data previously recorded in the memory, the RFID tag can also allow the registration of new data in the memory which overwrite the previous data or are additional to those data.

The RFID tag may also include other discrete electronic components such as capacitors, transistors, and diodes as well as logical elements for controlling the various components of the RFID tag.

The RFID tag may also include a battery or alternatively it may be of the passive type, in which case the antenna is used to provide power (to activate) the RFID circuit through an electromagnetic field provided by an interrogation device (reader) at the time of the interrogation of the RFID tag.

Within the scope of the present invention, the RFID tag is of the type operating in the UHF (Ultra High Frequency) band, thus with a circuit operating at a frequency preset in the UHF band. Preferably, the operative frequency is between 600 MHz and 1200 MHz.

The monitoring system according to the invention further comprises a device for interrogating comprising a reader and at least one interrogation antenna operatively associated to at least one RFID tag. In accordance with an aspect of the present invention, each interrogation antenna is arranged in a designated seat obtained in the support for the fruits and is located close to, preferably under, a respective cavity for containing a fruit or close to a group of containing cavities. Each interrogation antenna is capable of distributing a strong and substantially uniform electromagnetic field in a proximity detection area in such a way to interact with the RFID tags associated to one or more containing cavities and located in the vicinity.

Each interrogation antenna operates in the UHF band, as defined above, and is preferably an RFID near-field UHF antenna which is compact and has a thin shape, for example with the form of a loop or a printed patch.

In the present invention, each interrogation antenna is arranged inside the support in the designated housing seat and is operatively connected to an interrogation electronic component, such as a reader, located outside the support for fruits. In this regard, the support may be provided with connection means, such as one or more connection ports, to put in electrical communication each interrogation antenna with the reader, for example via designated connection cables which are connected to the opposite ends in respective ports of the reader and of an interrogation antenna, or through multiplexer connections wherein each multiplexer connection connects a plurality of interrogation antennas to the reader.

In the present invention, also the RF reader operates in the UHF band and can interrogate the RFID tags associated to the fruits through the interrogation antennas arranged in the support and recover the data from their memory by emitting a RF signal of interrogation compatible with the circuit of each RFID tag which is such to “activate” said circuit and to produce a response signal for each RFID tag through the transponder which is read and recorded by the reader.

The reader may be of the fixed type or advantageously of the portable type.

It has been surprisingly found that by using a system of measurement based on RFID technology with a fixed setup, that is to say wherein the RFID tags associated to the cavities for containing fruits and the interrogation antennas of the device for interrogating said RFID tags are maintained in a fixed position and at a short distance from each other, in particular close to each other, one achieves an improvement of accuracy and/or precision of the single measurements of electromagnetic signals, for example in frequency and/or power, reflected by the RFID tags and related to the state or degree of ripeness of the respective fruits.

Moreover, advantageously, by using a plurality of RFID tags associated to each containing cavity and therefore to each fruit, it is possible to obtain more electromagnetic signals of response of the RFID tags from each fruit and in different areas from the same, allowing to improve the precision of prediction of the state or degree of ripeness of each fruit.

As regards the method for monitoring the degree of ripeness of a plurality of fruits according to the invention, in a preliminary step the fruits to monitor are each arranged in a respective containing cavity of the support according to the invention and to each of the fruits is associated at least one RFID tag arranged in contact with or close to the respective fruit. In this regard, the RFID tags may be preemptively arranged in the respective containing cavities, for example attached on the bottom and/or on the side walls delimiting said cavity, thereby constituting part of the support according to the invention so that, in use, with the fruits arranged in the respective containing cavities the above-mentioned RFID tags are in contact with or close to the respective fruits, preferably in contact with said fruits. Alternatively, the RFID tags may be directly attached on the fruits whose state or degree of ripeness is to be monitored, in one or more areas of the same, for example a basal area and/or one or more side areas of the fruit, preferably equatorial areas. In the present invention, mixed arrangements comprising RFID tags attached to the fruits and RFID tags arranged in the respective cavities for containing said fruits are also considered.

Subsequently, an electromagnetic field (or signal) in the UHF band is generated by the reader which is irradiated by each interrogation antenna so as to run over one or more RFID tags operatively associated thereto, said RFID tag or tags being in turn associated to one or more containing cavities close to said interrogation antenna. Each electromagnetic field irradiated by an interrogation antenna involves then essentially the RFID tags of the containing cavities associated thereto. Said electromagnetic field or signal may have a preset frequency and/or phase and/or power or may be in the form of a scan within a preset range of frequency and/or phase and/or power.

From each RFID tag associated to a cavity for containing a fruit is therefore obtained a backscattered/reflected electromagnetic signal of response towards the respective antenna whose frequency and/or phase and/or power is affected by the peculiar dielectric characteristics of the fruit at issue that change over time in connection to the changes the fruit undergoes due to ripening (moisture, acidity, sugars) or to physiological alterations (texture) that occur during its storage. Consequently, each response signal deviates from the expected response signal obtainable in the absence of the perturbation of the dielectric environment around the RFID tag which is due to the presence of the fruit at issue.

Said deviation translates in an electromagnetic signal transmitted (i.e. reflected/backscattered) by each RFID tag to the interrogation antenna associated thereto, wherein each transmitted electromagnetic signal has a modified frequency and/or phase and/or power related to the degree of ripeness of a respective fruit or, in other words, in a variation of the frequency and/or phase and/or power (or radiation efficiency) with respect to the preset value, which variation is related to or can be related to the degree of ripeness of the fruit at issue on which said RFID tag is directly or indirectly applied.

The transmitted signals are recorded by the respective reading antennas and by the reader, and the data relative to or related to the frequency and/or phase and/or power of the transmitted signal and/or the data relative to or related to the variation of frequency and/or phase and/or power between the transmitted signal and a reference value of frequency and/or phase and/or power recorded by the reader are processed by the processing unit so as to produce a piece of information more readily “readable” and preferably suitable to be displayed which is related to the state or degree of ripeness of each fruit located in the containing cavities of the support. The reference values of frequency and/or phase and/or power may be set in the RFID tags or stored in a database in communication with the processing unit.

In this regard, it is possible to use classification/learning algorithms, supervised or non-supervised, which, starting from the transmitted data, distinguish the various levels of ripeness of the single fruits.

The processing may be implemented by suitable electronic means/components which use one or more of the above algorithms and are located directly in the fixed reader or in the portable reader. Alternatively, the data detected by the reader may be transmitted and processed by an external electronic device, for example a computer or a smartphone.

At the end of the processing, an information response about the state of ripeness of single fruits is obtained, which may be suitably displayed in a local or remote display device, for example a screen of any electronic device. In particular, it may be displayed using a screen of the same reader device.

The information may be in the form of any graphic composition (writing, graphics and/or images, etc.) that can be identified by the user, whether he/she is a logistics-responsible employee or a final consumer, as representative of the state of ripeness of the fruit.

In another embodiment, the information may be provided in the form of a QR code which is preferably dynamic and preferably shows all the relevant or desired information about the tracking and ripeness of the fruits and is capable of connecting to the web.

According to an embodiment of the present invention, the processed data relative to or related to the frequency and/or phase and/or power of the signals transmitted by said RFID tags may be compared, for example by a reader or an electronic device, with reference signal data relative to or related to an initial state before the ripening of the fruit at issue. Said reference data may be contained in a database for each fruit of interest or they may be assessed on the fruits to monitor, also using the system according to the invention, before the ripening of the same, for example right after the harvest or the positioning of said fruits in a ripening cell.

From this comparison, an information response for each fruit related to the variation between the processed data relative to said fruit and reference data is obtained, and said information response can be used to classify the single fruits according to their state or degree of ripeness and said classification of each fruit may be displayed by the display means.

In particular, the reference data may be classified, for each type of fruit, into classes of ripeness of the fruit at issue and the information response obtained after the comparison of the data relative to or related to the frequency and/or phase and/or level of power of the signal transmitted to said reader and processed by the processing means with the reference data may be the association and the display of the class they belong to which corresponds to each reading made by the RFID reader.

For example, the reference data may be classified, for each fruit, into three classes which respectively correspond to the situation wherein the fruit is not ripe yet, it is in suitable/optimal ripening conditions for consumption or has an altered texture such as to limit/compromise its consumption, and the information response corresponding to one of these situations and displayed by the display means can be that of a fruit belonging to the class “unripe”, “ripe” or “very ripe”, respectively. In an embodiment of the invention, each class of classification of the fruits can be subdivided in subclasses for a more functional classification of the fruit according to particular or specific needs. For example, the class “ripe” may comprise subclasses which are functional for the proper placement of the fruit in the distribution chain, such as “stock”, “grocery” and “consumer”.

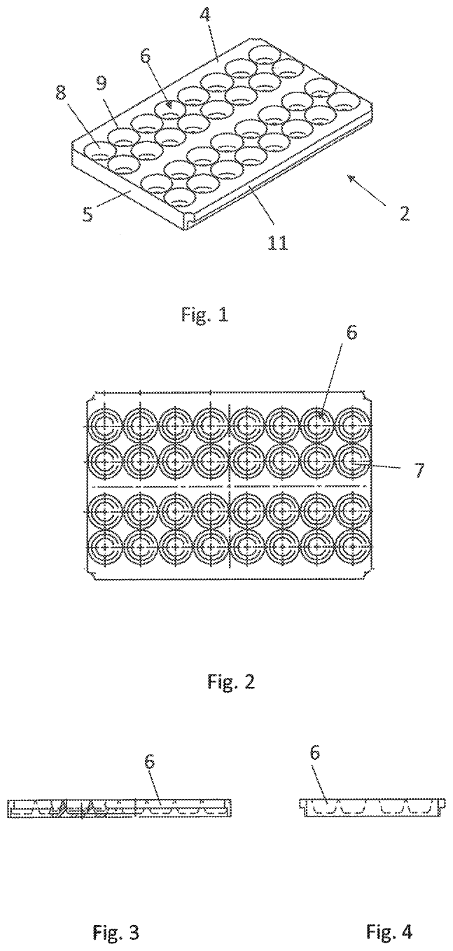

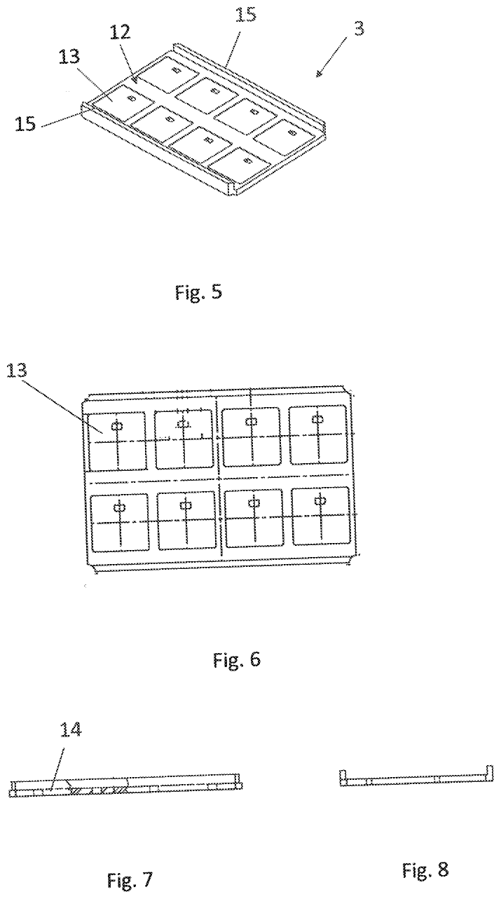

FIGS. 1-10 show a support for a plurality of fruits which is suitable for being used a system for monitoring the state or degree of ripeness of said fruits according to non-limiting embodiments of the present invention.

The support comprises a tray 2 and a base 3 that are intended to be removably coupled with each other with the tray 2 arranged on the base 3.

More in detail, the tray 3 has a substantially rectangular form and comprises an upper wall 4 and side walls 5 which extend downward from said upper wall 4. The tray 3 further comprises a plurality of cavities 6 suitable for containing a plurality of fruits, one for each fruit 10, which are preferably integrally formed with the material of the tray 3 and extend downward from said upper wall 4.

Each cavity 6 is delimited by a bottom wall 7 and at least one side wall 8 that ends in an upper edge 9 which defines the cavity opening 6. The cavities 6 have suitable sizes such as to preferably partially contain/house the fruits to monitor associated thereto, i.e. an upper portion of each fruit 10 protrudes at the top from a respective containing cavity 6 when arranged therein.

In the present embodiment, each cavity 6 has substantially the shape of a vase of small height and circular section with a diameter which is smaller on the bottom 7 and increases from the bottom 7 up to the opening 9. Obviously, the containing cavities 6 can also have different shapes, for example with square or rectangular section.

In the present embodiment, the containing cavities 6 are arranged one after the other along parallel rows extending in the longitudinal direction of the tray 2 and are substantially adjacent to each other in each of said rows.

In accordance with an aspect of the present invention, to each cavity for containing a fruit 10 is associated at least one RFID tag. Said at least one RFID tag may be arranged, for example, on the bottom 7 and/or on the side walls 8 of each containing cavity 6 and/or directly on the fruit 10 arranged in said containing cavity 6.

The tray 2 further comprises a pair of protrusions 11 on two opposite sides of the same, in this instance the long sides of the tray 2, which extend outwards from the upper wall 4 as an extension of the latter. The protrusions 11 extend substantially along the whole length of the respective opposite sides of the tray 2 and can be advantageously used as grip elements for the user for handling the tray 2.

The base 3 has a substantially rectangular shape comprising a lower portion 12 in which a plurality of housing seats 14 are obtained which are suitable for containing respective interrogation antennas 13. In particular, the interrogation antennas 13 are arranged one after the other in the respective housing seats 14 along parallel rows that extend in the longitudinal direction of the lower portion 12 of the base 3 so as to be located substantially under and close to one or more containing cavities 6 with the tray 2 arranged on the base 3 and coupled thereto.

In this way, each interrogation antenna 13 can be operatively associated to the RFID tags associated to one or more cavities 6 for containing respective fruits located close thereto, with said RFID tags being arranged in contact with or close to said fruits (for example arranged in the containing pits 6 and/or directly attached to the respective fruits).

For example, in the present embodiment, each interrogation antenna 13 can be operatively associated to the RFID tags associated to four adjacent containing cavities 6 arranged in opposite pairs on adjacent rows.

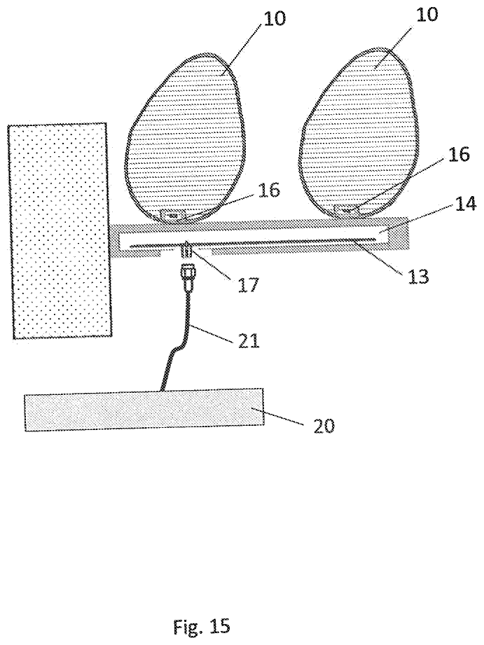

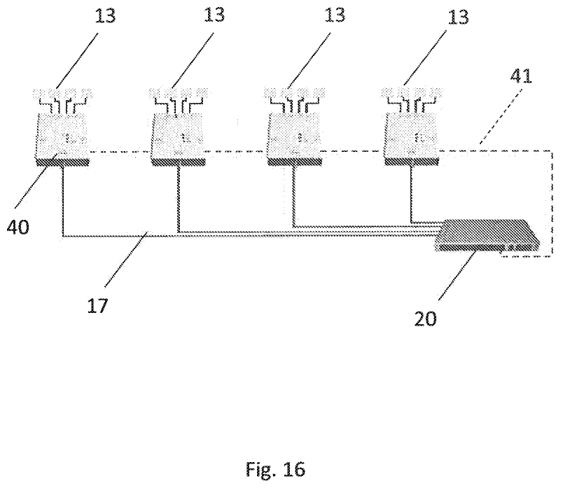

Moreover, the housing seats 14 containing the respective antennas 13 have each an aperture at a lower side of the lower portion of the base 3 at which connection means are arranged, for example a port 17. This allows to put each antenna 13 in electrical communication with a reader 20, for example via a cable 21, as schematically illustrated in FIG. 15. Alternatively, the antennas 13 can be connected in groups to the reader 20 via a multiplexer 40 and moreover an ethernet connection cable 41 may be provided, as schematically illustrated in FIG. 16.

In this way, each antenna 13 can irradiate an electromagnetic field (or signal) in the UHF band generated by the reader 20, that runs over one or more RFID tags operatively associated to the antenna and associated to one or more cavities for containing respective fruits, and can transmit the response signals received from said RFID tags and related to the state or degree of ripeness of said fruits toward the reader 20 for processing and subsequent displaying.

The base 3 further comprises a pair of protrusions 15 which extend upwards from the opposite lateral ends of the lower portion 12 of the base 3, in this instance from the ends of the long sides of the base 3.

In the present embodiment, the lower portion 12 of the base with the upper protrusions 15 has a substantially complementary shape to that of the tray 2, so as to allow to house said tray 2 in a seat delimited by the inner walls of the protrusions 14 and by the upper wall of the lower portion of the base 2 with the protrusions 11 which extend towards the outside of the tray 2 abutting on the upper part of the upper surface of the protrusions 15 oriented toward the top of the base 3.

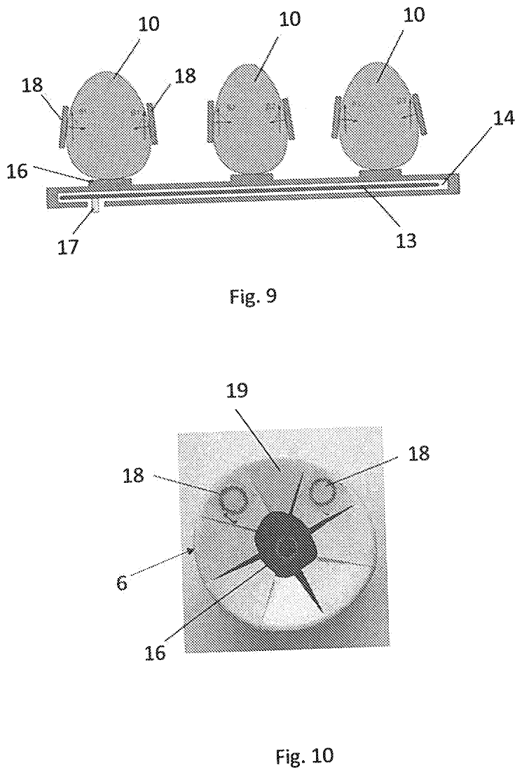

FIG. 9 schematically shows an example of arrangement of RFID tags associated with respective containing cavities (the tags being not shown in FIG. 9) of the support according to the invention.

In such example, with each containing cavity is associated a plurality of RFID tags comprising a first tag 16 arranged, in use, in contact with the base of a respective fruit 10, and second tags 18 arranged, in use, in contact with or close to a respective side area of said fruit 10, in this instance a central or equatorial area. In this case, the first tag 6 may be arranged (for example attached) on the bottom 7 of a containing cavity 6 while the second tags 18 may be arranged on the side walls 8 of the containing cavity 6, said RFID tags 16, 18 thereby constituting integral part of the support according to the invention. Alternatively, the RFID tags 16, 18 may be attached, in use, directly to the respective fruits, with the first tag 16 attached to the base of the fruit 10 and the second tags 18 attached at the side areas, for example in an equatorial area, of said fruit.

The second tags 18 may be arranged perpendicularly with respect to a transverse plane which crosses the fruit 10, for example in the equatorial area, or with a preset predefined angle with said plane, as illustrated for example in FIG. 9.

FIG. 10 schematically shows another example of arrangement of RFID tags associated with a containing cavity according to another embodiment of the support according to the invention.

In such example, each containing cavity 6 of the support further has, along the whole perimeter of the opening 8, a succession of tongues 19 folded inwards, i.e. that extend from the opening 8 of the containing cavity 6 toward the inside thereof. Said tongues advantageously facilitate the insertion of the fruit 10 in the respective containing cavity 6 thereby ensuring the latter to be centered and/or maintained in position by interference with said fruit 10.

Moreover, in this example, with each containing cavity 6 is associated a first RFID tag 16 arranged (for example attached) on the bottom 7 of the containing cavity 6 and second tags 18 arranged (for example attached) on respective tongues 19, said first RFID tag 16 and said second RFID tags coming, in use, in contact with a respective fruit 10 arranged in said containing cavity 6. This has the advantage to facilitate loading and unloading the fruits from the cavities 6.

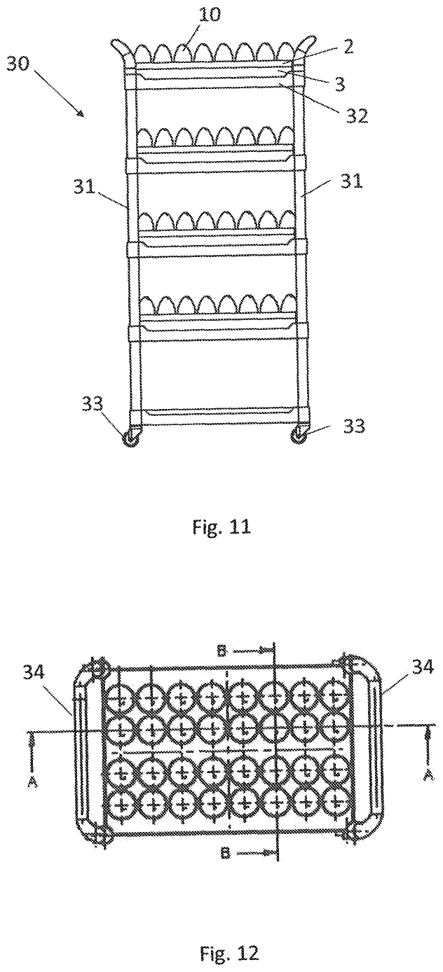

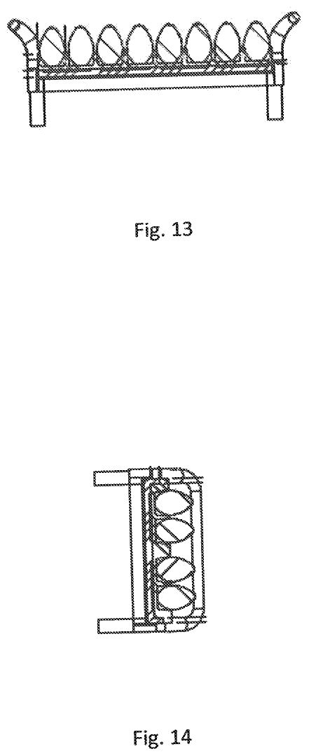

FIGS. 11-14 show a stillage according to an embodiment of the invention, wherein said stillage is referred to as a whole with reference number 30.

The stillage 30 comprises a frame with four tubular vertical supporting elements 31 and a plurality of shelves 32 arranged between the supporting elements 31 at predetermined heights between each other and fixed to said supporting elements 31 by suitable fixing means. In particular, in the present embodiment, the tubular elements 31 pass through respective holes (not shown) formed closed to the corners of the shelves 32 and are provided with conventional anchorage systems which insert between the holes of the shelves 32 so as to maintain them in position.

The shelves 32 have each a bearing seat adapted to receive a respective support comprising a tray 2 and a base 3 as previously described.

Moreover, the stillage 32 has four small wheels 33 fixed each to the lower end of a respective vertical supporting element 31 so that it is advantageously transportable for example by an operator.

The stillage has also a pair of opposite handles 34 fixed each to the upper ends of two opposite supporting elements 31.

A stillage as described above in association with respective supports according to the invention which contain fruits in the respective containing cavities can be advantageously used, for example, in a ripening chamber for monitoring the state or degree of ripeness of said fruits by constantly performing readings, over a preset time period, of the signals transmitted by the at least one RFID tag associated with each containing cavity (and therefore with each fruit), after activation, to the respective interrogation antenna and therefore to an external reader which processes said data and displays a piece of information related to the state of ripeness of each of said fruits.

In an embodiment, the stillage 30 comprises, integrated, a reader of the RFID tags associated to the containing cavities of the supports, said processing means (for example a computer) and said display means. This advantageously allows to transport the whole monitoring system in a simple way, thanks to the movable stillage, as needed.

In another embodiment, the reader 20 of the RFID tags associated to the containing cavities 6, the above-mentioned processing means and the above-mentioned display means are instead delocalized with respect to the stillage 30 and are operatively connected to each other and to the supports containing the cavities 6 for containing fruits during the use of the system according to the invention. For example, the stillage 30 with the supports containing the fruits to monitor can be placed in a fixed way in a chamber for ripening the fruits and the reader 20 of the RFID tags, the above-mentioned processing means and/or the above-mentioned display means may be arranged outside the ripening chamber and operatively connected, in use, to each other and to the supports containing the cavities for containing fruits.

In the light of the above, the RFID system according to the invention and the method for its use in monitoring the state or degree of ripeness of a plurality of fruits achieve the predetermined purposes and have several advantages.

In fact, thanks to the fact that the monitoring system according to the invention uses a fixed setup with interrogation antennas arranged in a fixed way close to the RDID tags associated therewith, the variability of the electromagnetic signal of response transmitted by the RFID tags to the reader is reduced and the signal-noise ratio (SNR) is improved, thereby obtaining a significant improvement of the accuracy and/or of the precision of the single measurements of electromagnetic signals, for example in frequency, phase and/or power, reflected by the RFID tags and related to the state or degree of ripeness of the respective fruits.

Moreover, advantageously, by using a plurality of RFID tags associated to each containing cavity and therefore to each fruit, it is possible to obtain more electromagnetic signals of response of the RFID tags from each fruit and in different areas from the same, allowing to improve the precision of prediction of the state or degree of ripeness of each fruit. In this regard, it is also possible to intercept the ripening of the fruits at an early stage, thereby anticipating the monitoring step.

Moreover, advantageously, the system according to the invention allows for continuous measurements or measurements at preset intervals over a preset time period on a plurality of fruits, for example of a production batch or over the whole process of fruit ripening in a ripening chamber, thereby obtaining a situation which is substantially in “real time” and representative of the state or degree of ripeness of all the above-mentioned fruits. It is also worth noting that the use of the monitoring system according to the invention does not require interruptions in the functioning of the process of ripening in chamber.

The monitoring system according to the invention is also effective and reliable and, at the same time, it can be produced at relatively low costs since RFID tags do not require special detection sensors and moreover no expensive analyzers and/or electronic components are required for its production. In any case, the RFID tags, if it is required, can also envisage the presence of sensors for specific needs, for example, for detecting other parameters related or not related to the fruit ripening, such as for example temperature, humidity, etc.

Moreover, the monitoring system according to the invention provides rapid readings of the degree of ripeness on a plurality of fruits, thereby being very convenient and practical in use. In this regard, it is also worth noting that the use of RFID tags integral with the containing cavities of the support makes the operations of loading and unloading the fruits from the respective cavities of the support rapider and more efficient.

In the light of the above, the system and the method for monitoring the degree of ripeness of a plurality of fruits according to the invention are very suitable for the use in the supply chain (storage, transport, over-the-counter sale, etc.) as well as in the scope of post-harvest monitoring and of monitoring the process of ripening in chamber with the purpose to preserve as long as possible the quality of fruit products, especially tropical fruits. In this way, waste is reduced and it is ensured that the fruits reach the market when they are at the proper degree of ripeness.

In order to meet particular and specific needs, a skilled in the art can make several modifications and variants to the system and the method for monitoring the degree of ripeness of a plurality of fruits according to the invention, wherein said modifications and variants are all within the scope of protection of the attached claims. For example, although the monitoring system of the invention has been described with reference to the detection of the degree of ripeness of fruits, it is evident that said system can be applied in the same way or in a similar way to any perishable food in so far as said food has peculiar dielectric characteristics that change over time in connection to the changes and/or physiological alterations that said food may undergo during its storage.

1. A support for monitoring the state or degree of ripeness of a plurality of fruits, in particular tropical fruits, comprising
a plurality of cavities for containing respective fruits, one cavity per each fruit,
a plurality of RFID tags associated to each cavity for containing a fruit and arranged, in use, in contact with or close to said fruit,
and a housing seat containing a respective interrogation antenna, each interrogation antenna being, in use, operatively associated to said plurality of RFID tags.

2. The support according to claim 1, wherein said plurality of tags comprise a first RFID tag arranged, in use, in contact with or close to a basal area of said fruit, and a second RFID tag arranged, in use, in contact with or close to a side area of said fruit.

3. The support according to claim 1, wherein each containing cavity has a bottom and a side wall ending in an upper edge or opening with perimeter extension and, optionally, a tongue extending from said upper edge or opening toward the inside of said containing cavity, and a plurality of RFID tags associated to each cavity for containing a fruit, said plurality of tags comprising a first RFID tag applied on the bottom of said containing cavity, and a second RFID tag applied on said side wall and/or on said tongue of said containing cavity.

4. The support according to claim 1, comprising a base and a tray removably associated or coupled with each other, the tray comprising said cavities for containing respective fruits, and the base comprising a housing seat and a respective interrogation antenna housed in said seat, each housing seat with the respective interrogation antenna being arranged close to, optionally under, a respective group of cavities for containing fruits and each interrogation antenna being, in use, operatively associated to the RFID tags associated to the containing cavities of the respective group.

5. A system for monitoring the degree of ripeness of a plurality of fruits, in particular tropical fruits, comprising
a support comprising:
a plurality of cavities for containing respective fruits, one cavity per each fruit,
a plurality of RFID tags associated to each cavity for containing a fruit and arranged, in use, in contact with or close to said fruit,
and a housing seat containing a respective interrogation antenna, each interrogation antenna being, in use, operatively associated to said plurality of RFID tags,
wherein the RFID tags associated to each cavity for containing a fruit operate in the UHF band,
the system further comprising:
an interrogation device for interrogating said plurality of RFID tags, comprising a reader, which is in electrical communication with said interrogation antenna, and
means for processing electromagnetic signals or data relative to or related to the degree of ripeness of said fruits coming from the reading of said RFID tags by said interrogation device.

6. The system according to claim 5, wherein said plurality of RFID tags associated to each cavity for containing a fruit comprise a first RFID tag arranged, in use, in contact with or close to a basal area of said fruit, and a second RFID tag arranged, in use, in contact with or close to a side area of said fruit.

7. The system according to claim 5, wherein each containing cavity is delimited by a bottom and a side wall ending in an upper edge or opening with perimeter extension and, optionally, it has a tongue extending from said upper edge or opening toward the inside of said containing cavity.

8. The system according to claim 7, wherein each containing cavity comprises a first RFID tag applied on the bottom of said containing cavity, and a second RFID tag applied on said side wall and/or on said at least one tongue of said containing cavity.

9. The system according to claim 5, wherein said support comprises a base and a tray which are removably associated or coupled with each other, the tray comprising said cavities for containing respective fruits, and the base comprising a housing seat and a respective interrogation antenna housed in said seat, each housing seat with the respective interrogation antenna being arranged close to a respective group of cavities for containing fruits and each interrogation antenna being operatively associated to the RFID tags associated to the containing cavities of the respective group.

10. The system according to claim 5, further comprising local or remote display means which are in communication with the processing means for displaying information about the degree or state of ripeness of fruits related to the data processed by the processing means.

11. The system according to claim 5, wherein said processing means are integrated in said reader.

12. A method for monitoring the degree of ripeness of a plurality of fruits comprising the steps of:
providing a support according to claim 1;
arranging a plurality of fruits in respective containing cavities of said support with a plurality of RFID tags operating in the UHF band and associated to each containing cavity, wherein said RFID tags are arranged, in use, in contact with or close to the respective fruit,
generating an electromagnetic signal in the UHF band by means of the reader of said interrogation device, each electromagnetic signal generated by said reader being irradiated/directed by an interrogation antenna so that it involves the RFID tags of a cavity for containing a fruit which is/are operatively associated to said interrogation antenna,
recording, by means of said interrogation device, a plurality of signals transmitted by the RFID tags associated to the respective cavities for containing the fruits, each signal being related to the frequency and/or phase and/or power transmitted by the RFID tags associated to a respective cavity for containing a fruit,
processing data relative to or related to the frequency and/or phase and/or power of the transmitted signals and/or processing data relative to or related to the variation of frequency and/or phase and/or power between the signals transmitted by the RFID tags and a preset value of frequency and/or phase and/or power,
displaying a piece of information about the degree or state of ripeness of said fruits related to the processed data.

13. The method according to claim 12, further comprising:
comparing the processed data relative to or related to the frequency and/or phase and/or power of the signals transmitted by said RFID tags with reference signal data relative to or related to an initial state before the ripening of said fruits, and
classifying said fruits according to their state or degree of ripeness on the basis of the variation between said processed data and said reference data,
displaying a piece of information about the classification of said fruits.

14. A stillage comprising a frame with vertical supporting elements, a plurality of shelves arranged between said supporting elements at predetermined heights between each other and connected to said supporting elements, and a plurality of supports according to claim 1 arranged each on a respective shelf.

15. The stillage according to claim 14, further comprising a plurality of small wheels fixed at the lower end of said vertical supporting elements and, optionally, a handle element, fixed at the upper ends of opposite supporting elements.

16. The stillage (30) according to claim 14, comprising, integrated, a reader of the RFID tags associated to the containing cavities of the supports, means for processing electromagnetic signals or data relative to or related to the degree of ripeness of fruits coming from the reading of said RFID tags, said processing means and local or remote display means in communication with the processing means for displaying information about the degree or state of ripeness of fruits related to the data processed by the processing means.
